(19) United States
(12) Patent Application Publication
(10) Pub. No.: US 2022/0015619 A1
(43) Pub. Date: Jan. 20, 2022

(54) PROTECTIVE SHIELD FOR MEDICAL PROCEDURES

(71) Applicant: Maurice Moshe Ernst, Jerusalem (IL)

(72) Inventor: Maurice Moshe Ernst, Jerusalem (IL)

(21) Appl. No.: 17/221,800

(22) Filed: Apr. 4, 2021

Related U.S. Application Data
(60) Provisional application No. 63/005,811, filed on Apr. 6, 2020.

(30) Foreign Application Priority Data
May 4, 2020 (IL) 27446

(51) Int. Cl. A61B 1/24; A41D 13/11; A61B 1/04; A61B 5/00

(52) U.S. Cl. CPC A61B 1/24; A61B 5/742; A61B 1/04; A41D 13/11

(57) ABSTRACT

A mask for covering the nose and mouth of a patient which supports a camera with a field of view into the oral cavity, and includes apertures for allowing for the passage of instrumentation into the oral cavity. The camera transmits images to a display, for example, in real time, through which the practitioner can see the area in the oral cavity where the procedure is being performed.

### CROSS-REFERENCES TO RELATED APPLICATIONS

This application is related to and claims priority from commonly owned U.S. Provisional Patent Application Ser. No. 63/005,811, entitled: Protective Mask For Medical Procedures, filed on Apr. 6, 2020

### TECHNICAL FIELD

The present disclosure is directed to personal protective equipment.

### BACKGROUND

When performing medical procedures on patients who are highly contagious, such as those with COVID-19, the working environment must be safe for the care staff. Such protection has gone as far as care staff wearing Haz Mat Suits. However, this is a bulky and expensive option, making procedures such as those inside the mouth difficult and time consuming.

For example, in the case of prevention of infection of the patient from accumulated plaque or bacterial clumps around the oral tissues and the intubation tubes, care workers typically use a toothbrush or other mechanical device. This causes emission of thousands of saliva droplets from the infected patient into the environment, transmitting the virus to all those in proximity to the infected patient. Under situations of highly transmitted pathogens and diseases such as the Covid-19 infection is transmitted to the medical staff who are reticent to clean the oral cavity and approximate areas because of self-infection. The resulting bacterial accumulation in the oral cavity, ora-pharynx, or pharynx can cause pneumonia or septacemias or other diseases. Therefore it is highly advantageous to have an apparatus that will isolate the oral cavity during medical procedures.

WO 2018/140841 A1 discloses a collapsible face mask including a sleeve(s) (e.g. for placement of an intubating blade) and/or other instruments.

EP 3512418 discloses a diagnostic tool for measuring an amount of nasal collapse in a patient. The diagnostic tool includes a mask with an endoscope port through which an endoscope which includes a camera is received. The mask also includes an opening to allow air flow, and an air flow sensor adapted to measure an inhalation rate of the patient.

### SUMMARY

The present disclosed subject matter provides a closed safe environment with non transmission for care staff when caring for highly infectious patients, such as those, e.g., the Corona virus patients. The disclosed subject matter is useful when performing any treatment via the oral cavity or the nose, where there is a chance of infection transmission. The treatments may include dental treatments, orthodontic treatments, regular mold or three dimensional (3D) digital impressions, dental surgery, prosthetics, implants, oral cleaning including dental cleaning, oral surgery, general surgery, intubation tube cleaning and maintenance and repositioning, and other procedures with entrance via or in the oral cavity or nose. The disclosed subject matter includes a patient worn face mask or mask, which accommodates the instrumentation and a camera useful in performing oral cavity procedures. The mask seals to the skin of the face, and allows the care worker full access to the oral cavity, allowing for full manipulations of the various working tools, without limitations of having to wear special suits, such that the care worker can work in standard personal protective equipment.

Embodiments of the disclosed subject matter are directed to a protective mask. The mask comprises: a body configured to cover at least the oral cavity; and, a camera positionable on the body so as to have a field of view including the oral cavity, for capturing images of the oral cavity.

Optionally, the protective mask is such that it additionally comprises: at least one aperture in the body for the passage of instrumentation therethrough.

Optionally, the protective mask is such that the camera is configured to capture video images.

Optionally, the protective mask is such that the camera is in communication with a display.

Optionally, the protective mask is such that the communication is wireless, and may be, for example, by Bluetooth™.

Optionally, the protective mask is such that the display includes at least one of: a smartphone, a monitor, or a computer.

Optionally, the protective mask is such that the body is configured to cover the oral cavity and the nose.

Optionally, the protective mask is such that the body is a self-supporting dome.

Optionally, the protective mask is such that the body is of a transparent material.

Optionally, the protective mask is such that the at least one aperture includes a sealing material for creating a seal with an instrument which passes through the at least one aperture.

Optionally, the protective mask is such that the at least one aperture in the body includes a plurality of apertures in the body.

Optionally, the protective mask is such that the camera is integral with the body, and positioned on the body to be in alignment with the oral cavity.

Optionally, the protective mask is such that it additionally comprises: at least one heating element of at least one of a battery, chemical, or solar power source, for heating the mask to avoid fogging of the mask.

Optionally, the protective mask is such that the at least one heating element includes one or more electrically conducting wires for heating to at least above body temperature.

Optionally, the protective mask is such that it additionally comprises: at least one coating for the inner side of the body which is at least one of: antifogging, insulating, anti-reflective, or an optical coating.

Optionally, the protective mask is such that it additionally comprises: a plurality of coatings along an inner side of the body.

Optionally, the protective mask is such that it additionally comprises: a micro mover for moving an intubation tube.

Embodiments of the disclosed subject matter are directed to a method for preforming a procedure in an enclosed surgical field. The method comprises: placing a mask, configured to cover a surgical field including at least the oral cavity and the nose of a human, over at least the nose and mouth of a human, the mask including a body, a camera positionable on the body so as to have a field of view including the surgical field, for capturing images of the surgical field, and including at least one aperture in the body for the passage of instrumentation therethrough; and, passing at least one instrument through the at least one aperture to perform the procedure.

Optionally, the method is such that the procedure includes at least one of: dental treatments, orthodontic treatments, regular mold impressions, three dimensional (3D) digital impressions, dental surgery, prosthetics, implants, oral cleaning including dental cleaning, oral surgery, general surgery, intubation tube cleaning and maintenance and repositioning, and other procedures with entrance through one or more of the oral cavity or nose.

Unless otherwise defined herein, all technical and/or scientific terms used herein have the same meaning as commonly understood by one of ordinary skill in the art to which the disclosure pertains. Although methods and materials similar or equivalent to those described herein may be used in the practice or testing of embodiments of the disclosed subject matter, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE DRAWINGS

Some embodiments of the disclosed subject matter are herein described, by way of example only, with reference to the accompanying drawings. With specific reference to the drawings in detail, it is stressed that the particulars shown are by way of example and for purposes of illustrative discussion of embodiments of the disclosed subject matter. In this regard, the description taken with the drawings makes apparent to those skilled in the art how embodiments of the disclosure may be practiced.

Attention is now directed to the drawings, where like reference numerals or characters indicate corresponding or like components. In the drawings:

FIG. 1 is a front view of the apparatus as worn by the patient;

FIG. 2 is a cross sectional view of the apparatus as worn by the patient, taken along line 2-2, the apparatus shown as part of a system, and showing a care worker performing a procedure in the oral cavity;

FIG. 3 is a front view of the apparatus in accordance with another embodiment, as worn by the patient;

FIG. 4 is a cross sectional view of the apparatus as worn by the patient, taken along line 4-4, the apparatus shown as part of a system, and showing a care worker performing a procedure in the oral cavity;

FIG. 5A is a cross sectional view of the mask of FIGS. 3 and 4 in use with an intubation tube; and,

FIG. 5B is cross sectional view of the intubation tube of FIG. 5A.

### DETAILED DESCRIPTION OF THE DRAWINGS

The disclosed subject matter is directed to a mask for covering the nose and mouth of a patient. The mask supports a camera with a field of view into a surgical or treatment field, such as the oral cavity, as well as body cavities, and includes apertures for allowing for the passage of instrumentation into the oral cavity, or body cavity. The instrumentation can be passed through mouth or the nose. The camera transmits images to a display, for example, in real time, through which the practitioner can see the area in the oral cavity or body cavity where the procedure is being performed.

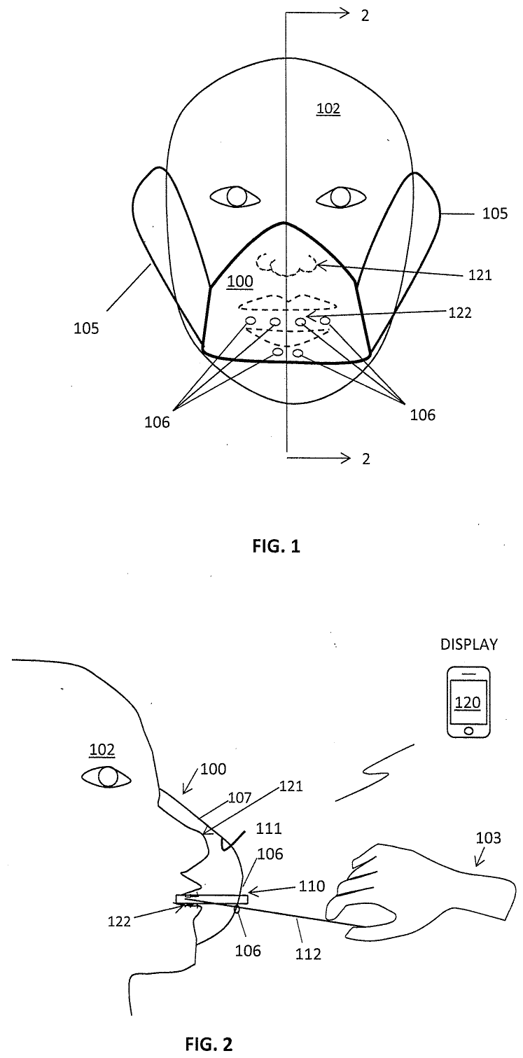

FIG. 1 shows the mask 100 as worn by the patient 102. The mask 100 serves to isolate the infected environment around the patient, e.g., the nose and mouth of the patient 102, while allowing the caregiver access to the nose and mouth, via instruments, including a camera, without the risk of contacting pathogens, germs, viruses, harmful agents, and the like, which are stopped by the mask 100. This allows the caregiver 103 to perform necessary medical treatments and functions without hindrance.

The mask 100 is placed on the skin of the face via mechanical placement, mechanical suction or adhesives at the edges, forming a seal, keeping pathogens from leaving the masked area. Additional attachment of the mask 100 to the face is facilitated by lines 105, that attach the mask 100 around the ears. The mask 100 includes a body portion or body 107, configured to cover at least the nose 121 and mouth 122. The body 107 is made of standard mask materials such as polyester, polymeric fibers, plastics, silicones and the like, and may also be of a transparent material, for example, either of a collapsible material or formed into a predetermined configuration, such as a dome shape, so as to be self-supporting when placed onto the face. The mask 100, for example, may be sterilizable, by autoclaving and the like.

Turning also to FIG. 2, the mask 100 includes openings or apertures or even a closed transparent window 106 for a camera 110 and instruments 112. The openings or apertures 106, extending through the mask 100, allowing for the passage of the camera 110 or instruments 112. The openings 106 are positioned on the mask 100 to allow for the camera 110 and instruments 112, such as tooth brushes, cleaning tools, dental drills, syringes, suction, dental tools, restorative dental tools, intra-oral video cameras, cameras, intra-oral three dimensional (3D) cameras, dental tools for filling teeth, orthodontic tools, intubation tubes, and, endoscopes to be manipulated easily to perform the various procedures in the oral or body cavity, The tools may also be integral to the mask. Additional instruments include a micro movement tool, or micro mover, for moving intubation tubes, for cleaning, repositioning and the like. These tools can be controlled manually, remotely or programmed for automatic use. By moving the intubation tube, with the micro mover, at regular or random time intervals, ulceration and infection is prevented. While the instruments 112 are typically removable, they may also be integral with the mask 100.

For example, the openings 106 are positioned proximate to the mouth allow for the camera 110 to align with the mouth. The openings 106 are, for example, single or double sealed with material 106x, such as elastomeric material, which closes on the instrument 112, contacting the instrument 112, to seal the instrument 112 (e.g., creating a seal with the instrument 112) from the outside. Additional or fewer openings 106 may be on the mask 100, at different positions, to accommodate the instrumentation for the requisite procedure. The apertures 106, while shown rounded or circular, may also be of other shapes, such as rectangular, triangular, oval, or combinations thereof.

The camera 110 may be, for example, an intra oral video camera, video camera, or other camera for capturing still images. The camera 110 is for example, separate from the mask 100, and extended therethrough as an instrument, or may be mounted and held on the mask 100, so as to be removable. Alternately, the camera 110 may be integral with the mask 100. The mask 100 is such that the opening for the camera 110 or camera mounts are positioned on the mask 100 such that the camera field of view aligns with the oral cavity.

The camera 110 allows for viewing of the oral cavity via a display 120, with the camera 110 linked to the display 120 either wired or wirelessly. The camera 110, for example, includes an antifogging lens, so should the mask 100 become fogged or steamed, the care worker can view the oral cavity on the display 120, and continue with the procedure. The display 120 is, for example, a smart phone, a monitor or the like. Alternately, when the mask 100 is transparent, the camera 110 may be outside or inside of the mask and joined to the mask 100 by mechanical fasteners, adhesives, and combinations thereof.

Additional means can be added to enable manipulation of the intubating tube so that it can be moved to prevent pressure ulcers or to enable cleaning. For example, small movements of the intubation tube can reposition the tube with relationship to the soft tissues and relive pressures that cause pressure ulcers. This can be done manually or possibly automatically by mechanical “arms” that grip the tube external to the mouth and are attached to the mask and manipulate the tube at preset or preprogrammed intervals.

The mask 100 may be of an insulating material, allowing the inner surface 111 (of the inner side of the mask 110), or portions thereof, of the mask 100 to be preheated, for example, to above body temperature (37 degrees C.), as an anti-fogging mechanism. The mask 100 may also include a defogging element, formed of embedded wires for conducting energy, the wires integrated into the mask 100, which attach to a power source, for heating the mask 100, for example to above body temperature. The inner patient-facing side of the mask may also be coated with a surfactant or other coating, creating a dispersing surface for any saliva or other body fluids that may accumulate on the mask 100, or a coating preventing clouding of the mask 100, or a coating insulating the mask 100 for heat or electronically, or optical coatings such as an anti-reflective coating, or a filter coating for specific wavelengths or polarized light. The identical or different coating materials may also be applied in layers.

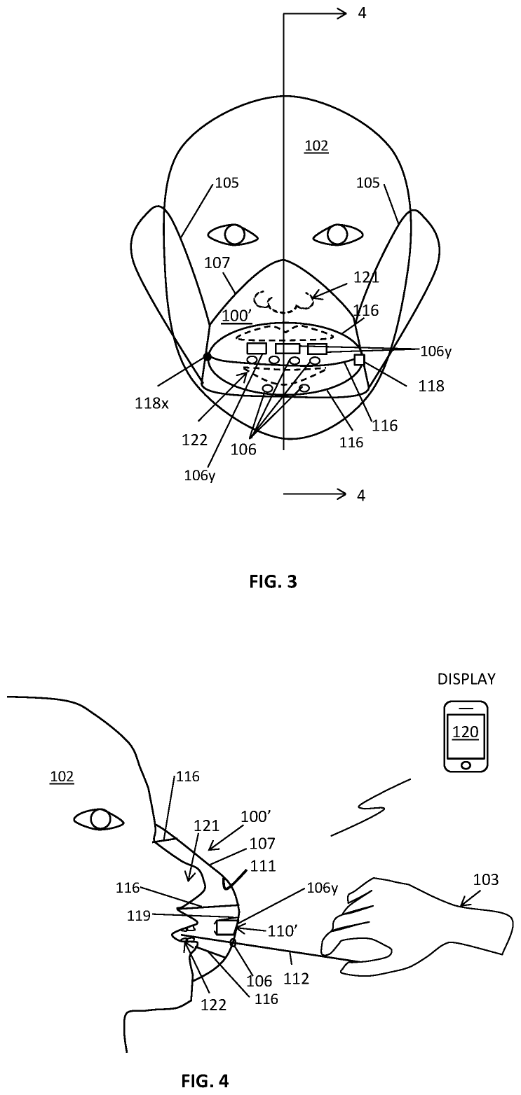

FIGS. 3 and 4 show an alternate embodiment 100′ of the mask 100, where like numbered elements to those in the mask 100 are in accordance with the descriptions provided for the mask 100 above. The mask 100′ includes apertures 106 as well as windows 106y associated with an integral camera 110′, or for intubation tubes and the like, positioned on the body 107 of the mask 100′ (inner side), held in or prepackaged (and sealed) into a pocket 119, so as to be aligned with the oral cavity 122. The pocket 119, may be open from one or both sides, either lateral, horizontal/vertical or combinations thereof. The camera 110′ is in wireless communication, for example, Bluetooth™, with the display 120, so as to transmit the captured images of the oral cavity, for example, real time video, used for the performance of the procedure. Other video and still images may also be captured by the camera 110′ and transmitted to the display 120. While one integral camera 110′ is shown, multiple integral cameras 110′, as well as other removable cameras 110 (also in wireless communication with the display 120), can be used. The windows 106y, while shown square or rectangular, may also be of other shapes, such as rounded, circular, oval, triangular, or combinations thereof.

The mask 100′ also includes heating elements 116, for example, electrically conducting wires, embedded in the body 107 (the material of the body 107 serving as an insulator), the wires, extending from a source 118, to a point 118x on the opposite side of the mask 100′. The source 118 is, for example, a battery, solar, or chemical energy source, which can be activated and deactivated to cause the wires 116 to heat, for example, to a range just over body temperature (body temperature is approximately 37 degrees Celsius in a healthy individual but can vary according to the time of day and health of the individual).

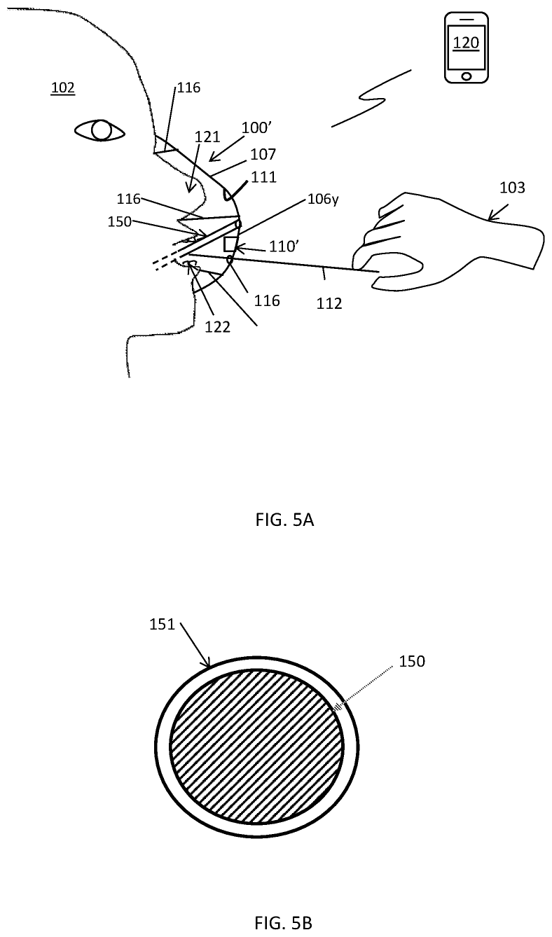

FIG. 5A shows the mask 100′ in use with an intubation tube 150. The intubation tube 150, is, for example, a rounded cylindrical tube, as shown in FIG. 5B. The components of the mask 100′ are in accordance with those shown in FIGS. 3 and 4, and as described above. The intubation tube 150 may be moved manually or automatically preprogrammed in time and movement by instruments 112, passing through the apertures 106, the instruments including a micro mover, or by a micro mover mounted internally on the body 107, or manually or automatically via the cover sleeve 151 around the intubation tube 150, or externally, via a mounted micro mover, or a manually operated micro mover.

The masks 100, 100′ are usable in numerous dental and surgical procedures, as performed in oral and/or body cavities. These dental and surgical include, for example, dental treatments, orthodontic treatments, regular mold impressions, three dimensional (3D) digital impressions, dental surgery, prosthetics, implants, oral cleaning including dental cleaning, oral surgery, general surgery, intubation tube cleaning and maintenance and repositioning, and other procedures with entrance through one or more of the oral cavity or nose.

It is appreciated that certain features of the disclosure, which are, for clarity, described in the context of separate embodiments, may also be provided in combination in a single embodiment. Conversely, various features of the disclosure which are, for brevity, described in the context of a single embodiment, may also be provided separately or in any suitable sub-combination.

It will be appreciated by persons skilled in the art that the present disclosed subject matter is not limited to what has been particularly shown and described hereinabove. Rather the scope of the present disclosed subject matter is defined by the appended claims and includes both combinations and sub-combinations of the various features described hereinabove as well as variations and modifications thereof, which would occur to persons skilled in the art upon reading the foregoing description.

1. A protective mask comprising:
a body which is self-supporting and configured for placement on the face to cover at least the oral cavity; and,
a camera mount integral with the body, and positioned on the body so as receive a camera such that the camera is oriented to include a field of view including the oral cavity, for capturing images of the oral cavity.

2. The protective mask of claim 1, additionally comprising: at least one aperture in the body for the passage of instrumentation therethrough.

3. The mask of claim 1, wherein the camera is configured to capture video images.

4. The mask of claim 1, wherein the camera is in communication with a display.

5. The mask of claim 4, wherein the communication is wireless.

6. The mask of claim 4, wherein the display includes at least one of: a smartphone, a monitor, or a computer.

7. The mask of claim 2, wherein the body is configured to cover the oral cavity and the nose.

8. The mask of claim 7, wherein the body is in the shape of a dome.

9. The mask of claim 8, wherein the body is of a transparent material.

10. The mask of claim 2, wherein the at least one aperture includes a sealing material for creating a seal with an instrument which passes through the at least one aperture.

11. The mask of claim 10, wherein the at least one aperture in the body includes a plurality of apertures in the body.

12. The mask of claim 1, wherein the camera when received by the mount is positioned to be in alignment with the oral cavity.

13. The mask of claim 1, additionally comprising: at least one heating element of at least one of a battery, chemical, or solar power source, for heating the mask to avoid fogging of the mask.

14. The mask of claim 13, wherein the at least one heating element includes one or more electrically conducting wires for heating to at least above body temperature.

15. The mask of claim 1, additionally comprising: at least one coating for the inner side of the body which is at least one of: antifogging, insulating, anti-reflective, or an optical coating.

16. The mask of claim 1, additionally comprising: a plurality of coatings along an inner side of the body.

17. The mask of claim 2, additionally comprising: a micro mover for moving an intubation tube.

18. A method for diagnosing a condition in the oral cavity, comprising:
placing a mask on the face of a human to cover at least the oral cavity and the nose of the human, over at least the nose and mouth of a human, the mask including a self-supporting body, a camera mount integral with the body, and positioned on the body so as to receive a camera in an orientation where the camera includes a field of view including the oral cavity, for capturing images of the oral cavity, and, at least one aperture in the body for the passage of instrumentation therethrough; and,
placing a camera into the camera mount to perform the diagnosing the condition.
